(19) United States
(12) Patent Application Publication
(10) Pub. No.: US 2008/0249197 A1
(43) Pub. Date: Oct. 9, 2008

(54) Process for the Production of Methanol from Methane using a Supported Transition Metal Catalyst

(76) Inventors: Maureen L Bricker, Buffalo Grove, IL (US); Timothy A. Brandvold, Arlington Heights, IL (US); Wensheng Chen, Hoffman Estates, IL (US); Shurong Yang, Elmhurst, IL (US); Joel T. Walenga, Lake Zurich, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/697,774

(22) Filed: Apr. 9, 2007

(51) Int. Cl. C07C 27/06

(52) U.S. Cl. 518/703; 514/703

(57) ABSTRACT

A process for the selective oxidation of methane to methanol using a supported transition metal catalyst has been developed. Examples of the transition metals which can be used are copper and palladium, while an example of a support is silica. Optionally, the catalyst can contain a modifier component such as cesium. Generally the process involves contacting a gas stream, comprising methane, a solvent such as trifluoroacetic acid and an oxidizing agent such as air or hydrogen peroxide with the catalyst, at oxidation conditions to produce a methyl ester, e.g. methyl trifluoroacetate. Finally, the methyl ester is hydrolyzed to yield a methanol product stream.

### FIELD OF THE INVENTION

This invention relates to a process for converting methane to methanol using a catalyst comprising a transition metal component such as palladium or copper and optionally a modifier component such as cesium dispersed on a support such as silica. Generally the process involves contacting a gas stream, comprising methane, a solvent such as trifluoroacetic acid and an oxidizing agent such as hydrogen peroxide or air with the catalyst, at oxidation conditions to produce a methyl ester, e.g. methyl trifluoroacetate. Finally, the methyl ester is hydrolyzed to yield a methanol product stream.

### BACKGROUND OF THE INVENTION

Today, both chemical and energy industries rely on petroleum as the principal source of carbon and energy. Methane is underutilized as a chemical feedstock, despite being the primary constituent of natural gas, an abundant carbon resource. Factors limiting its use include the remote locations of known reserves, its relatively high transportation costs and its thermodynamic and kinetic stability. Methane's main industrial use is in the production of synthesis gas or syngas via steam reforming at high temperatures and pressures. Syngas in turn can be converted to methanol also at elevated temperatures and pressures. The production of methanol is important because methanol can be used to produce important chemicals such as olefins, formaldehyde, acetic acetate, acetate esters and polymer intermediates. The above two step process for the production of methanol is expensive and energy intensive with corresponding environmental impacts.

Selective oxidation of methane has been studied for over 30 years by individual, academic and government researchers with no commercial success. For example, Sen et al. in New J. Chem, 1989, 13, 755-760 disclose the use of Pd (O₂C Me)₂ in trifluoroacetic acid for the oxidation of methane to CF₃CO₂Me. The reaction is carried out for 4 days at a pressure of 5516-6895 kPa (800-1000 psi). E. D. Park et al. in Catalysis Communications, Vol. 2 (2001), 187-190, disclose a Pd/C plus Cu (CH₃COO)₂ catalyst system for the selective oxidation of methane using H₂/O₂ to provide H₂O₂ in situ. L. C. Kao et al. in J. Am. Chem. Soc., 113 (1991), 700-701 disclose the use of palladium compounds such as Pd (O₂CC₂H₅)₂ to oxidize methane to methanol in the presence of H₂O₂ and using trifluoroacetic acid as the solvent. U.S. Pat. No. 5,585,515 discloses the use of catalysts such as Cu(I) ions in trifluoroacetic acid to oxidize methane to methanol. WO 2004069784 A1 discloses a process for the oxidation of methane to methanol using transition metals such as cobalt or manganese in trifluoroacetic acid. Finally, M. N. Vargaftik et al in J. Chem. Soc., Chem. Commun. 1990(15) pp. 1049-1050 disclose results for a number of metal perfluoro acetate compounds. The metals which were found to be active were Pd, Mn, Co and Pb. Copper was found to have virtually no activity.

Applicants have developed a liquid phase process for the oxidation of methane to methanol. The process involves the use of a catalyst comprising a transition metal component such as a copper component and optionally a modifier component such as cesium dispersed on a support such as silica. This catalyst is contacted with an oxidizing agent such as hydrogen peroxide or air and methane gas all dissolved in a solvent such as trifluoroacetic acid under mild conditions to provide a methyl ester which is hydrolyzed to methanol.

### SUMMARY OF THE INVENTION

As stated, this invention relates to a process for converting methane to methanol comprising contacting a gas stream comprising methane with a catalyst comprising a transition metal component dispersed on a solid support, the transition metal is selected from the group consisting of manganese, copper, palladium, heteropoly acids, molybdenum, rhenium, iron, platinum, cobalt, silver and mixtures thereof in the presence of an oxidizing agent selected from the group consisting of air, oxygen, hydrogen peroxide, organic hydroperoxides and mixtures thereof and a solvent at oxidation conditions to provide a methyl ester compound and hydrolyzing the methyl ester compound at hydrolysis conditions to provide a methanol product stream. Examples of supports include silica, aluminas, silicon carbide, silica-alumina, molecular sieves etc. The catalyst can optionally contain a modifier component such as cesiun.

This and other objects and embodiments will become clearer after a detailed description of the invention.

### DETAILED DESCRIPTION OF THE INVENTION

The present invention relates to a liquid phase process for the oxidation of methane to methanol. One necessary component of this process is a catalyst which promotes the selective oxidation of methane. The catalyst comprises a transition metal component dispersed on an inorganic oxide support. The transition metals which can be used are selected from the group consisting of manganese, copper, palladium, heteropoly acids, molybdenum, rhenium, iron, platinum, cobalt, silver and mixtures thereof. Heteropoly acids are complex oxoanions of varying stoichiometry containing a transition metal such as molybdenum and tungsten and a base element such as silicon and phosphorous. The oxoanion charge is balanced either by proton or alkali, or alkaline earth cations. Optionally other metals including vanadium, and iron, can be partially substituted for the transition metal. Examples of common heteropoly acids include without limitation H₃[P(Mo₃O₁₀)₄] and K₆P₂W₁₈O₆₂.

The transition metal is dispersed on an inorganic oxide support which is selected from the group consisting of silica, silicon carbide, aluminas, silica-alumina, zirconia, titania, magnesium oxide, ceria, lanthanum oxide, aluminum phosphate, molecular sieves, and mixtures thereof. It should be pointed out that silica-alumina is not a physical mixture of silica and alumina but means an acidic and amorphous material that has been cogelled or coprecipitated. This composition is well known in the art; see e.g. U.S. Pat. No. 3,909,450; U.S. Pat. No. 3,274,124 and U.S. Pat. No. 4,988,659 all of which are incorporated by reference in their entirety. Molecular sieves include zeolites and non-zeolitic molecular sieves (NZMS). Examples of zeolites include, but are not limited to, zeolite Y, zeolite X, zeolite L, zeolite beta, ferrierite, MFI, mordenite and erionite. Non-zeolitic molecular sieves (NZMS) are those molecular sieves which contain elements other than aluminum and silicon and include silicoaluminophosphates (SAPOs) described in U.S. Pat. No. 4,440,871, ELAPOs described in U.S. Pat. No. 4,793,984, MeAPOs described in U.S. Pat. No. 4,567,029 all of which are incorporated by reference. Aluminas which can be used include without restriction gamma alumina, delta alumina, eta alumina and theta alumina.

Dispersion of the transition metal onto the support is accomplished by means well known in the art which includes impregnation, precipitation, ion exchange, and reductive deposition with impregnation being preferred. Impregnation is carried out by preparing a solution of a transition metal compound and then contacting the inorganic oxide with the solution for a time sufficient to absorb the transition metal compound onto the support. The transition metal compounds which can be used to prepare the solution include without limitation the oxide, hydroxide, nitrate, acetate, halides, oxalate, and acetylacetonate. In addition transition metal complexes which contain neutral or charged coordinating ligands can also be used. Specific examples of the transition metal compounds include without limitation Mn₂O₃, Mn₃O₄, MnO₂, KMnO₄, K₂Mn₄P₃O₁₆, MnPO₄.H₂O, Na₂Mn₂P₂O₉H₂O, KMn₈₀₁₆, (FeMn)PO₄, Mn(II)trifluoroacetate, Mn(II) acetate, Mn(III)acetate, CO₂O₃, Co(II) Acetate, AgO, Ag(I)trifluoroacetate, Fe₂O₃, copper nitrate, copper acetate, palladium acetate etc.

Water is the solvent which is usually used to prepare the solution although organic solvents such as ethanol or acetone can be used. Once the compound is absorbed onto the support, it is dried and then calcined at a temperature of about 100° C. to about 800° C. for a time of about 1 hour to about 48 hours. The resultant catalyst can be used in the form of a powder or a shaped article. Examples of shaped articles include without limitation spheres, pills, pellets, extrudates, irregularly shaped particles, etc. Means for preparing these shaped articles are well known in the art. Deposition of the transition metal compound can be done either before or after the powder is formed into a shaped article although not necessarily with equivalent results. Metal impregnation before forming is preferred. When a transition metal oxide is the desired compound, it can be deposited on an inorganic oxide by commingling it with the support and then forming it into a shaped article by means such as extrusion, marumerizing, pelletizing, etc. Depending on post synthesis treatment conditions the metal may be on the support as a metal cation, metal oxide, reduced metal, or a mixture thereof. Regardless of the form of the transition metal on the inorganic oxide support, the transition metal is present in an amount from about 0.1 wt. % to about 10 wt. % of the catalyst as the metal.

The support can optionally have dispersed thereon a modifier component selected from the group consisting of alkali metals, alkaline earth metals and mixtures thereof. Specific examples include without limitations potassium, cesium, lithium, sodium, magnesium, calcium, strontium and barium. The modifier component can be impregnated onto the support in the same manner as described for the catalytic metals. In this regard the compounds which can be used to prepare a solution of the metal include without limitation the oxides, hydroxides, nitrates, halides, acetate, oxalate, acetylacetonate, etc. Specific examples include KNO₃, CsNO₃, sodium hydroxide, magnesium acetylacetonate dihydrate, calcium iodide, strontium nitrate, and barium acetate. The modifier component can be impregnated onto the support before, after or simultaneously with the catalytic metal although not necessarily with equivalent results. The preferred impregnation order is dependent on the specific metals, counterions, supports and reaction conditions needed to prepare the target material. For example if copper and potassium are the catalytic metal and modifier respectively and the support is silicon carbide and the compounds are the nitrate salts, then co-impregnation is preferred. Also, as stated above the oxide form of the modifier metal can be used and incorporated as stated for the catalytic metal oxides above. Finally, post synthesis treatment conditions will determine if the modifier metal is present on the support as the metal cation, metal oxide, reduced metal or a mixture thereof. Regardless of the form in which the modifier component is present on the support, it is present from about 0.1 to about 10 wt. % as the metal.

Another necessary component of the invention is a solvent which acts as the reaction medium. Non limiting examples of solvents are trifluoroacetic acid, trifluoroacetic anhydride, pentafluoropropionic acid, acetic acid, supercritical carbon dioxide, sulfuric acid, sulfur trioxide, trifluoromethanesulfonic acid, methanesulfonic acid and mixtures thereof with trifluoroacetic acid being preferred. Another necessary ingredient of the process is an oxidizing agent selected from the group consisting of air, oxygen, hydrogen peroxide, organic hydroperoxides and mixtures thereof. Examples of organic hydroperoxides include but are not limited to tert-butylhydroperoxide, cumene hydroperoxide, etc. When oxygen is the desired oxidizing agent, it can be used as pure oxygen or blended with inert diluents. Diluents which can be used include without limitation nitrogen, argon, helium, etc. In the oxygen/diluent blends, the amount of oxygen can vary widely but is usually between 5 and 30 volume percent. If air or other gaseous oxidizing agents are used, then the oxidizing agent is typically added to the reaction mixture directly from a compressed gas cylinder or tank or via atmospheric source with a mechanical compressor. The amount of oxidizing agent present in the solvent can vary over a wide range, but usually varies from about 0.1 mol % to about 50 mol %. This mixture is now placed into a pressure vessel to which is added a methane stream in a concentration sufficient to produce a pressure of about 103 kPa (15 psi) to about 6895 kPa (100 psi) and preferably from about 4137 kPa(600 psi) to about 6895 kPa (100 psi). The pressurized reaction vessel is now heated at a temperature of about 25° C. to about 250° C. and preferably from about 60° C. to about 100° C. The vessel is held at this temperature for a time of about 30 minutes to about 24 hours in order to contact the methane with the oxidizing agent, catalyst and solvent and provide a mixture comprising a methyl ester formed from the methane and an adduct from the solvent. Additional oxidizing agent can be periodically added, i.e. intermittent addition, to obtain higher conversion of methane to the methyl ester.

The methyl ester formed, such as methyl trifluoroacetate, can be separated from the reaction mixture by any suitable methods but distillation is preferred. The methyl ester, e.g. methyl trifluoroacetate (MTFA) is now hydrolyzed to produce free methanol and regenerate the solvent. Using MTFA as an example, although it is understood that the process is not limited to MFTA, the MFTA is introduced into a hydrolysis reactor along with water. The amount of water introduced is at least the stoichiometric amount required for complete hydrolysis although it is preferred to use an excess amount of water. A catalyst and a co-solvent may also be used. A variety of acidic and basic substances are known to promote ester hydrolysis. Suitable acids include but are not limited to hydrochloric acid, sulfuric acid, trifluoroacetic acid, toluene sulfonic acid, acidic alumina, silica-alumina, sulfated zirconia, and acidic ion exchange resins. Suitable basic materials include but are not limited to sodium hydroxide, lithium hydroxide, potassium hydroxide, and solid bases such as hydrotalcite. Acid hydrolysis is preferred to allow easy recovery of the trifluoroacetic acid solvent/product. When hydrolysis is complete the methanol product can be separated from the reaction mixture by a variety of methods known in the art including distillation, adsorption, extraction and diffusion through a membrane. Separation of trifluoroacetic acid is achieved by analogous methods. The recovered trifluoroacetic acid is then recycled to the oxidation reactor.

In addition to carrying out the process in a batch mode as described above, the process can also be conducted in a continuous mode as follows. The catalyst is placed in a fixed bed high pressure reactor and the methane, oxidizing agent and solvent flowed through the bed at the temperatures and pressures set forth above. Methane, oxidizing agent and solvent may be added independently to the reactor or mixed prior to introduction to the reactor. The solvent/methane/oxidizing agent mixture is flowed through the catalyst bed at a liquid hourly space velocity (LHSV) of about 0.1 hr⁻¹ to about 100 hr⁻¹. Gas and liquid are removed from the reactor continuously at a rate to maintain the liquid level and total pressure in the reactor. The removed gas/liquid stream is transferred to a vessel where the gas and liquid are separated and one or both streams may be subjected to further separation or returned to the high pressure reactor.

### EXAMPLE 1

### Synthesis of 1% Cu/1% K/SiC

To a container containing 0.2334 g of a 5% Cu(NO₃)₂ solution and 1.2284 g of a 0.95% KNO₃ solution there were added 1.167 g of SiC support. The resulting impregnated catalyst was dried and calcined at 400° C. for 6 hours. This catalyst was identified as catalyst A.

### EXAMPLE 2

A sample of catalyst A was tested for methane oxidation as follows. To a glass liner containing 57.1 mmol of trifluoroacetic anhydride and 100 mg of catalyst A at a temperature of −20° C. there were added 10.6 mmol of a 36% hydrogen peroxide solution. The mixture temperature was maintained at below 0° C. during the addition of the peroxide. The glass liner was then put into an 80 cc Parr™ autoclave and the reactor quickly assembled and pressurized with 4238 kPa (600 psig) of 95% methane with 5% Argon as an internal standard. The autoclave was then held at 80° C. for 3 hours. After the 3 hours, the liquid sample was analyzed by both NMR and GCMS and the gas sample was analyzed by GC equipped with FID, TCD and MS detectors. The estimated methane based yield was calculated based on methanol product (isolated as methyl trifluoroacetate) divided by methane introduced into the system. Methanol product was calculated based on GCMS analysis or NMR analysis, and the amount of methane introduced into the system was based on the weight difference before and after the introduction of methane gas and ideal gas law occasionally. Analysis showed that 1.47% methanol product was formed.

### EXAMPLE 3

Catalyst A (300 mg) was tested as in Example 2 except that the reactor was heated to 100° C. for ½ hour. Analysis showed that 1.50% methanol was produced.

### EXAMPLE 4

A methane oxidation test was run as in example 2 except that 180 mg of spent catalyst A from example 3 was used. Analysis showed that 1.28% methanol was produced.

### EXAMPLE 5

The spent solution from example 3 was tested as per example 2 without any additional catalyst but with the addition of 15.9 mmol of a 50% hydrogen peroxide solution. Analysis showed that 1.77% methanol was produced.

### EXAMPLE 6

### Synthesis of 5% Cu/1% K/5% Cs/SiC Catalyst

About 200 (±5%) mg of SiC support was placed into a miniature reaction well and to it there were added 273 μl water, 197 μl of a 5% Cu(NO₃)₂ solution, 52 μl of a 4% KNO₃ solution, and 227.5 μl of a CsNO₃ solution. The impregnated catalyst was then dried and calcined at 400° C. for 6 h. This catalyst was identified as catalyst B.

### EXAMPLE 7

To a 300 cc Parr reactor equipped with gas entrainment impeller there were added 500 mg of catalyst B, 40 ml of trifluoroacetic acid and 81 ml of trifluoroacetic anhydride. The reactor was assembled, pressurized with methane to 4238 kPa (600 psig) and then ramped to 100° C. and kept there for 3 hours, during which time 309 mmol of a 50% hydrogen peroxide solution were added using an ISCO pump at an addition rate of 0.145 ml/min for 2 hours. After the 3 hour period the liquid and gas samples were analyzed as in example 2. These analyses showed that 28.6% methanol product formed. The calculated catalyst turn over number (TON) was 195.

### EXAMPLE 8

### Synthesis of 5% Mn/SI1254 Catalyst

About 200 (±5%) mg of Davison silica (SI1254) support were placed in a miniature reaction well and to it there were added 427 μl of water and 323 μl of a 3.1% Mn(NO₃)₂ solution. The impregnated catalyst was then dried and calcined at 600° C. for 6 h. This catalyst was identified as catalyst C.

### EXAMPLE 9

To an 80 cc Parr reactor there were added 300 mg of catalyst C and 10 ml of trifluoroacetic acid. The reactor was assembled and pressurized first with methane to 4238 kPa (600 psig), then with 8% oxygen in nitrogen to give a final pressure of 6996 kPa (1000 psig). The reactor was heated at 180° C. for 3 hours. The liquid sample and gas samples were analyzed as in example 2. These analyses showed that 4.08% methanol product was formed.

### EXAMPLE 10

To a 300 cc Parr reactor equipped with gas entrainment impeller there were added 150 ml of trifluoroacetic acid and 600 mg of catalyst C. The reactor was assembled and pressurized first with methane to 700 kPa (100 psig), then with 8% oxygen in nitrogen to 4238 kPa (600 psig). The reactor was heated at 180° C. for 3 hours. The liquid and gas samples were analyzed per example 2. These analyses showed that 27.1% methanol product was formed. The calculated catalyst TON was 53.

### EXAMPLE 11

### Synthesis of 5% Mn/Grace 59 Catalyst

About 200 (±5%) mg of silica support (Grace 59) were placed into a miniature reaction well and to it there were added 430 μl water and 320 μl of a 3.1% Mn(NO₃)₂ solution. The impregnated catalyst was then dried and calcined at 600° C. for 6 h. This catalyst was identified as catalyst D.

### EXAMPLE 12

To an 80 cc Parr reactor were added 10 ml of trifluoroacetic acid and 300 mg of catalyst D. The reactor was assembled and pressurized first with methane to 4238 kPa (600 psig), then with 8% oxygen in nitrogen to give a final pressure of 6996 kPa (1000 psig). The reactor was heated at 180° C. for 3 hours. The liquid and gas samples were analyzed as per example 2. These analyses showed that 3.48% methanol product was formed.

1. A process for converting methane to methanol comprising contacting a gas stream comprising methane with a catalyst comprising a transition metal component dispersed on a solid support, the transition metal is selected from the group consisting of manganese, copper, palladium, tungsten, molybdenum, rhenium, iron, platinum, cobalt, silver and mixtures thereof in the presence of an oxidizing agent selected from the group consisting of air, oxygen, hydrogen peroxide, organic hydroperoxides and mixtures thereof and a solvent at oxidation conditions to provide a methyl ester compound and hydrolyzing the methyl ester compound at hydrolysis conditions to provide a methanol product stream.

2. The process of claim 1 where the oxidation conditions comprise a temperature of about 80° C. to about 200° C., a pressure of about 103 kPa(15 psia) to about 6867 kPa (1000 psia), a contact time of about 1 min to about 24 hrs and an oxidizing agent concentration from about 0.1 mol % to about 50 mol%.

3. The process of claim 1 where the solid support is selected from the group consisting of silica, silicon carbide, aluminas, silica-alumina, zirconia, titania, magnesium oxide, ceria, lanthanum oxide, aluminum phosphate, molecular sieves and mixtures thereof.

4. The process of claim 1 where the hydrolysis conditions include a temperature of about 20° C. to about 200° C. and a pressure of about 103 kPa (15 psi) to about 1030 kPa (150 psi) and at least a stoichiometric amount of water.

5. The process of claim 1 further comprising carrying out the hydrolysis in the presence of a catalyst selected from the group consisting of acidic catalysts and basic catalysts.

6. The process of claim 5 where the acidic catalyst is selected from the group consisting of hydrochloric acid, sulfuric acid, trifluoroacetic acid, toluene sulfonic acid, acidic alumina, silica-alumina, sulfated zirconia, acidic ion exchange resins and mixtures thereof.

7. The process of claim 5 where the basic catalyst is selected from the group consisting of sodium hydroxide, lithium hydroxide, potassium hydroxide and hydrotalcite.

8. The process of claim 1 where the oxidizing agent is hydrogen peroxide.

9. The process of claim 1 where the oxidizing agent is intermittently added.

10. The process of claim 1 where the solvent is selected from the group consisting of trifluoroacetic acid, trifluoroacetic anhydride, pentafluoropropionic acid, acetic acid, supercritical carbon dioxide, sulfuric acid, sulfur trioxide, trifluoromethanesulfonic acid, methanesulfonic acid and mixtures thereof.

11. The process of claim 1 where the process is a batch process.

12. The process of claim 1 where the process is a continuous process.

13. The process of claim 1 where the transition metal is copper.

14. The process of claim 3 where the support is silica.

15. The process of claim 1 where the catalyst further comprises a modifier component selected from the group consisting of alkali metals, alkaline earth metals, and mixtures thereof.

16. The process of claim 15 where the modifier component is selected from the group consisting of potassium, cesium, lithium, sodium, magnesium, calcium, strontium and barium.

17. The process of claim 15 where the modifier component is present on the catalyst from about 0.1 to about 10 wt.% as the metal.

18. The process of claim 1 where the oxidizing agent is oxygen.

19. The process of claim 18 where the oxidizing agent is oxygen blended with a diluent selected from the group consisting of argon, nitrogen, helium and mixtures thereof.
